(19) United States
(12) Patent Application Publication
(10) Pub. No.: US 2021/0138454 A1
(43) Pub. Date: May 13, 2021

(54) CARRIER FOR CELL CULTURING AND A METHOD OF PREPARATION THEREOF

(71) Applicant: LYOPOR s.r.o., Brno (CZ)

(72) Inventors: Josef Jancar, Brno (CZ); Lucy Vojtova, Menin (CZ)

(73) Assignee: LYOPOR, s.r.o., Brno (CZ)

(21) Appl. No.: 16/680,887

(22) Filed: Nov. 12, 2019

(51) Int. Cl. B01L 3/00; G01N 33/483; C12N 5/00

(52) U.S. Cl. CPC B01L 3/5025; G01N 33/4833; C12N 2537/10; B01L 2300/0829; C12N 5/0068

(57) ABSTRACT

A carrier for cell culturing that contains a multiwell plate, where at least one of the wells of the multiwell plate has a porous substrate having the porosity of ≥90% and adapted for cell culturing, and the porous substrate adheres to the surface of the well. A method for preparation of the carrier for cell culturing is also provided.

### FIELD OF ART

This invention relates to a solid carrier containing a 3D substrate suitable for cell culturing and analyses of properties of any cell type. The invention further relates to a method of preparation of such solid carrier.

### BACKGROUND ART

Cell culturing has emerged as one of the key tools used in molecular and cellular biology to study cell physiology, effects of toxins and various drugs on cells, and biochemistry, mutagenesis, and carcinogenesis of cells. Cell and tissue cultures are also used for the large scale production of biological compounds such as therapeutic proteins and vaccines, and for the screening and development of a variety of therapeutic drugs. The advantage of being able to obtain consistent results from the use of a batch of cultured cells continues to encourage the increased usage of cell and tissue culture for such applications.

The standard 2D cell culture systems grow cells on flat dishes, typically made of plastic. The cells are put onto coated surfaces where they adhere and spread. Unfortunately, 2D cell cultures do have some inherent flaws. Growing on a flat surface isn't a good way to understand how cells grow and function in a human body, where they surrounded by other cells in three dimensions. 2D cell testing isn't always predictive, which increases the cost and failure rate of new drug discovery and clinical trials. As cells grow in a standard 2D culture, they consume growth media and exude waste. This can result in toxic waste products, dead cells, nutrition depletion and damage of the environment the cells are in.

Despite these disadvantages, 2D cell cultures are still used for the majority of cell cultures. Economies of scale mean 2D cell culture systems and technologies are less expensive than some other systems. 2D cell cultures have been used since the early 1900s, gaining widespread acceptance in the 1940s and 1950s. It's easier to compare current results vs. previous results and studies. Everyone involved with lab work has been taught 2D cell culture technology. 2D cell cultures are typically easier to analyze than some 3D cell culture systems. Therefore, both the researchers and the biotech companies are increasingly leaning towards the use of 3D cell culture systems that allow the analysis of the interaction between different human cell types and the extra cellular matrix (ECM). The business with 3D cell cultures emerged over the last 10 years. The 3D cell culture industry is starting to catch up, especially in cancer research, drug testing and stem cell research. Reasons for the increasing acceptance and use of 3D cell cultures are multiple.

Much better biomimetic tissue models make 3D cell cultures more physiologically relevant and predictive than 2D cultures. Creation of complex systems linked together by microfluidics means that 3D tissue systems can better model how different types of cells interact. Fluid flow, for example blood flow, interstitial fluid flow and urine flow, is crucially important for the functioning of all tissues. Cells respond to flow through differentiation and metabolic adaptation. Epithelia typically separate organ compartments, interact with the environment and protect the organism from the environment. Their proper functioning is crucial for survival and barrier malfunction plays a role in many diseases. Representation of barrier tissues is greatly enhanced in 3D culture. Microfluidics continuously provide nutrients to where they're needed, meaning cells and organs grow in a more realistic way. Animal models aren't a reliable way to predict how drug treatments will affect humans. Screening drugs against human organs grown on chips is a much more reliable method. 3D cell culture systems are good simulators of diseased tissue, including cancer tumors. They can exhibit similar growth and treatment patterns.

A 3D substrate provides a biomimetic extracellular matrix (ECM), in which cells can survive, grow and proliferate. These bio-mimicking ECMs allow the passage of nutrients and gasses to give the cells the environment they need to thrive. Well-developed and selected ECMs also provide essential cues to cells, rendering them crucial for the establishment of physiologically relevant 3D tissue cultures. This means 3D cell cultures with ECMs can be accurately grown and measured, providing an ideal environment for drug discovery and development and other types of research.

The demand of the tissue engineering research, cancer research and pharmaceutical industry for a wide range of cultured cells has risen considerably and stimulated the development of manufacturing techniques for large-scale 3D substrate manufacturing. To become commercially successful, the products have to address multiple issues such as:
Throughput—Many 3D culture techniques are cumbersome and time-consuming, rendering them unsuitable for drug development screening and research.
Challenges in microscopy and measurement—Due to the larger size of 3D cell cultures compared to 2D cell cultures, some types of measurement and microscopic analysis can be difficult.
Getting oxygen and other essential nutrients to the right place—For larger 3D cultures, it can be a challenge to distribute nutrients to where they need to be in the cell culture.

So far, a large number of various 3D substrates has been introduced in the scientific literature, varying in chemical composition as well as by the preparation method. Substrates were reported made of polymers, biopolymers, bioceramics, bioactive glass, metals collonized by cells as well as from composites prepared by commination of organic and inorganic components. To achieve the desired biological functionalities, substrates were modified with various natural and synthetic bioactive species, growth factors, blood derivatives, functional proteins, etc., to enhance proliferation and/or differentiation of cells derived from a specific tissue or organ.

Biological experiments, drug and cosmetics testing, diagnostics, tissue engineering and cancer research using specific cell types are characterized by greatly varying results and poor transferability of the laboratory and test animal results to human. In addition to the cell variability and adaptability, poor reproducibility in substrate fabrication is among the most frequent causes of these discrepancies. Extremely poor reproducibility of the 3D substrate morphology made of polymers and biopolymers (collagen, cellulose, fibroin, etc.) reduces the reliability of cell essays in tissue engineering and cancer research substantially leading to non-effective utilization of state of the art analytical instruments capable of automatically process large number of specimens. The above shortcomings can be overcome substantially by discovering method for scaffold fabrication with highly reproducible 3D porous morphology controlled at macro-, micro- and nano-scales directly in the well of the standardized cell culturing well plate.

### DISCLOSURE OF THE INVENTION

Object of the invention is a carrier for cell culturing, which contains a multiwell plate, wherein at least one of the wells of the said multiwell plate contains a 3D (three-dimensional) porous substrate and wherein the said 3D porous substrate adheres to the surface of the said well. Such carriers are capable of mimicking extracellular matrix of a range of tissues for a highly reproducible culturing of a broad range of cell types.

The porous substrates are three-dimensional (3D), and not planar (2D), i.e., they have a thickness (a smallest dimension) of at least 10 micrometers, thus allowing cell growth in a volume, and not only on the surface, and also enabling vascularization.

The porous substrates are adapted for cell culturing due to their pore connectivity enabling transport of oxygen and nutrients to cells and transport of cell excrements away from the cells. The size and connectivity of the pores varies for different cell types and is derived from the morphology of the extracellular matrices of the hard and soft tissues it mimicks, thus ideally simulating the real tissue ECM environment for the cultivated cells. The size and the connectivity of the pores in the targeted tissue as well as in the cell culturing substrate are measured and compared employing generally known and verified methods.

The multiwell plate contains at least 2 wells, preferably at least 4 wells, more preferably at least 12 wells. The upper limit of the number of wells in the plate is determined by technical limitations, preferably it is 10,000 wells, more preferably 2,000 wells. The wells may exhibit a circular or non-circular cross section.

The porous substrate can be provided in at least one well of the multiwell plate, preferably in at least 25% of the wells, more preferably in at least 50% of the wells or in all the wells of the multiwell plate.

The multiwell plate is preferably made of a material selected from the group consisting of plastic, glass, ceramics, metals, and combinations of these materials.

Preferably, the pore size distribution of the porous substrates is the same in all the wells of the wellplate. Further preferably, the pores in the porous substrate are interconnected. It is further preferable if the substrate has a relative porosity ≥90%. More preferably, the substrates have the relative porosity between 95 and 99.5%, and most preferably having the relative porosity between 97 and 99.5%.

The porous substrate can consist of one or more layers. Preferably, the porous substrate is composed of at least two layers, each layer having a different porosity, pore size, pore connectivity, material composition, type and/or content of bioactive additives.

The pore size of the porous substrate is within the range from 0.1 to 1000 μm, preferably between 5 and 1000 μm or between 100 and 2000 nm or within the range from 50 to 600 μm.

The material of the porous substrate is preferably selected from the group consisting of proteins of the extracellular matrix (ECM); structural proteins such as collagens, fibrin, silk fibroin, elastin or gelatine; polysaccharides such as hyaluronic acid and its derivatives, chitosan and its derivatives, starches, resinous gums, cellulose and its derivatives; resins; biocompatible synthetic polymers, such as polylactic acid, polyglycolic acid, polyethyleneglycol and their block copolymers, polycaprolaktone, polyhydroxybutyrate, polyurethanes; natural inorganic nanotubes (such as Halloyzit, etc.); and combinations of these materials. Natural and synthetic polymeric materials and macromolecular systems can preferably be cross-linked, either reversibly or irreversibly. Material of the porous scaffold can be in the form of a xerogel foam, porous hydrogel, particles, fibers or nano-fibers. Material of the porous substrate can contain additives such as proteins, especially functional bioactive proteins, their fragments or mixtures, phosphates, organic or inorganic nano-particles such as hydroxyapatite, alpha- or beta-tricalcium phosphate, oxide ceramics, such as SiO₂, ZnO, metal nanoparticles such as Ag, Au, Mg, or metal nano-particles decorated with covalently or physically bound organic ligands. Nanoparticles are commonly identified as particles having at least one dimension in the interval between 0.1 až 1,000 nm. The substrate material composition can contain up to 50 wt. % of additives, relative to the dry content of the material of the porous substrate, preferably up to 30 wt. %, or up to 20 wt. %, or up to 10 wt. %, or from 0.1 to 5 wt. %.

The material of the porous substrate is preferably selected from the group consisting of collagen, collagen cross-linked with EDC/NHS, silk fibroin, homo- and copolymers of polylactic acid (PLA), polyglycolic acid (PGA) and/or polyethylene glycol (PEG), polycaprolaktone, polysaccharides such as chitosan, oxidized cellulose, gum-karaya, silk fibroin. Spinnable materials can be in the form of fibers or nano-fibers. Preferably, the material of the porous substrate contains ECM specific additives such as hydroxyapatite, oxidized cellulose, blood platelets, bioactive proteins from collagenous tissues, hyaluronic acid, polycaprolaktone, gelatine, growth factors, bone morphogenic proteins, haloisit.

The porous substrate has the thickness (height) of at least 10 micrometers, preferably at least 100 micrometers, more preferably at least 0.5 mm, most preferably between 1 and 10 mm.

A further object of the invention is a method for preparation of a carrier for cell culturing, the said method comprising the following sequence of steps:
applying a layer of solvent into at least one well of the multiwell plate and freezing the layer of solvent,
applying at least one layer of a material for a porous substrate into the said at least one well of the multiwell plate, and freezing each layer after it is applied,
applying a cover layer of the solvent into the said at least one well of the multiwell plate, followed by freezing the cover layer, and
freeze drying after applying each layer separately, or after applying all the layers into the said at least one well.

The material of the porous substrate is preferably in the form of a solution, suspension, aerogel, emulsion, hydrogel, microparticles or nano-fibers.

The presence of the solvent in the top and bottom layers enables to control homogeneity of the substrate surface during the freeze drying (lyophilization) process. The solvent layers are not present in the final product since they are removed in the process of freeze drying.

Water is the preferred solvent, more preferably, ultrapure water is the solvent of choice. Uniform, gradient or engineered pore size and size distribution is preferably achieved by pore nucleation and by placing the material of the substrate on top of the frozen water at the bottom of the well, followed by freezing the material of the porous substrate deposited in the form of solution or gel. The process is terminated by depositing the top layer of water on top of the layered system followed by final freeze drying step. Freeze drying can be carried out using various sublimation rates which allow to control size, size distribution and connectivity of pores.

Especially when using ultrapure water as the solvent, the freeze drying is preferably performed in two steps. First freezing to a temperature between −5 and −40° C., followed by freezing to a temperature between −20 and −60° C.

Freeze drying is performed preferably at a temperature lower than −70° C., preferably at a pressure lower than the atmospheric pressure; more preferably at a pressure of 1 kPa or less. Using water as the solvent, the freeze drying consists of the ice sublimating from capillaries and pores at a temperature and pressure lower than the critical point on the phase diagram of water, (i.e., critical temperature T_{K}=373,95 K and critical pressure p_{K}=22.06 MPa).

The material of the porous substrate can be cross-linked during the substrate preparation using cross-linking agents suitable for the given composition. A person of ordinary skill in the field will have sufficient knowledge about the appropriate cross-linker systems for any given polymeric component. For example, for collagen, EDC/NHS is a suitable biologically inert cross-linking system.

The method of preparation as described herein ensures that the substrate morphology is porous with uniform pore size, desired pore size distribution and pore connectivity in all wells of the solid carrier having thickness ranging from 10 μm up to the thickness equal to the individual well depth. The method further allows for adjusting the specific parameters of the production process to their optimized values, thus allowing to control porosity, pore size and size distribution as well as pore interconnectivity. The substrates according to this invention with reproducible pore size, size distribution and interconnectivity in all wells containing the substrate, enable imminent seeding of cells, thus providing conditions for obtaining highly reproducible and reliable results in biological and medical research, tissue engineering, diagnostics, drug and cosmetics testing and in procedures of regenerative medicine.

The process for preparation of the porous substrate includes a precisely controlled and reproducible procedure of layering biologically acceptable materials with a suitable porosity, pore size, size distribution and spatial organization of interconnected pores at high relative porosity (≥90%), enabling vascularization for transport of oxygen, nutrients and removal of waste, endowing favorable ECM-like living conditions for cells to proliferate and differentiate within the entire volume of the substrate. Such a substrate has not been prepared with the existing procedures, so far. The substrate exhibits long term shelf life at room temperature without significant loss of biological properties. The combination of the substrate with the carrier allows for simple manual or automated laboratory and clinical manipulation.

Further, the disclosed invention includes the use of the system for cell culturing, process of cell culturing and possible analyses of the cultured cell specimens.

### BRIEF DESCRIPTION OF DRAWINGS

FIG. 1 shows SEM micrographs of the surface of the surface of the porous 3D collagenous substrate. Left according to the example 1 (0.5 wt. % of collagen I) and right according to example 2 (combination of 0.5 a 2 wt. % collagen I layers).

FIG. 2 shows the composition dependence of the pore size of the 3D collagen I substrate (left), Effect of the HAP concentration at constant collagen I content (right).

FIG. 3 shows nanoCT scan of the porous substrate according to example 3 (grey area) and its 3D colonization by cells of lung cancer (dark grey circular objects).

FIG. 4 shows fluorescence confocal microscopy of differentiated MSC cells on the porous substrate from example 1. Survival rate of the cells is documented by staining with BCECF-AM/propidiumjodide 28 days after seeding the porous substrate containing 0.5 wt. % collagen I and (a) 50 wt. % nHAP, (b) 70 wt. % nHAP and (c) containing 2 wt. % collagen I and 50 wt. % of nHAP Immunofluorescence detection of osteocalcin 28 days after seeding the same substrates.

FIG. 5 shows SEM micrographs of the 3D substrate from example 6 (left) containing hyaluronic acid according and containing chitosan according to example 7.

FIG. 6 shows fluorescence confocal microscopy of differentiated cells on the 3D substrate from the example 8 (left) and 9 (right). Survival rate of cells is documented by staining their cytoplasm with green BCECF-AM/propidiumiodide and their nuclei in red with propidium iodide.

FIG. 7 shows fluorescence confocal microscopy of differentiated cells on the porous 3D substrate from example 10 (left) and 11 (right). Survival rate of cells is documented by staining their cytoplasm with green BCECF-AM/propidiumiodide and their nuclei in red with propidium iodide.

### EXAMPLES OF CARRYING OUT THE INVENTION

### Example 1

Wells in a 48-well plate were layer by layer filled with a biocompatible material in the following sequence:
1. Ultrapure water (Milipore, purity II)
2. Layers of homogeneous solutions of bovine collagen I, concentration ranging from 0.3 to 3 wt. % in ultrapure water
3. Ultrapure water (Milipore, purity II).

The materials in the wellplate were frozen under controlled conditions after application of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with pore size engineered to the range of 250-450 μm and thickness of 5 mm adhering to the well walls. Even after swelling, the substrate remains attached to the well bottom without detaching and floating (see FIG. 1). Porosity in the layers was proportional to the collagen concentration and was within the interval from 99.5% for the 0.3 wt. % collagen I concentration to 97% for collagen concentration of 3 wt. %. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value. This substrate is, thus, suitable for cell seeding and culturing and has already been subject to in-vitro and in-vivo tests with various cell types.

### Example 2

Wells in a 48-well plate were layer by layer filled with a biocompatible material in the following sequence:
1. Ultrapure water (Milipore, purity II)
2. Layer of homogeneous solutions of bovine collagen I, concentration of 2 wt. % in ultrapure water
3. Layer of homogeneous solutions of bovine collagen I, concentration of 0.5 wt. % in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 6 mm exhibiting controlled pore size of 100 μm in the bottom layer 3 mm thick as well as in the top layer 3 mm thick exhibiting pore size of 250-450 μm and adhering to the well walls. Even after swelling, the substrate remains attached to the well bottom without detaching and floating. This substrate is, thus, suitable for seeding and culturing broad range of cell types and has already been subject to in-vitro and in-vivo tests (see FIG. 2). Porosity in the bottom layer was equal to 98.5% for the 2 wt. % collagen I concentration and 99.3% for collagen concentration of 0.5 wt. %. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 3

Wells in a 96-well plate were layer by layer filled with a biocompatible material in the following sequence:
1. Ultrapure water (Milipore, purity II)
2. Layer of electrospun nanofibers consisting of the mixture of polycaprolactone and gelatine and having fiber diameter of 120-280 nm
3. Layer of homogeneous solutions of bovine collagen I, concentration of 0.5 wt. % in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 3 mm exhibiting controlled pore size of 50 μm in the bottom layer 0.5 mm thick as well as in the top layer 2.5 mm thick exhibiting pore size of 400 μm and adhering to the well walls. Even after swelling, the substrate remains attached to the well bottom without detaching and floating. This substrate is, thus, suitable for seeding and culturing broad range of cell types and has already been subject to in-vitro and in-vivo tests. Growth of the cells throughout the entire volume of the 3D substrate is depicted in FIG. 3. Porosity in the bottom layer was equal to 45.3% for nanofibrous layer and 99.3% for collagen concentration of 0.5 wt. %. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 4

Wells in a 48-well plate were layer by layer filled with a biocompatible material in the following sequence:
1. Ultrapure water (Milipore, purity II)
2. Layer of homogeneous solutions of bovine collagen I, concentration of 0.5 wt. % in ultrapure water
3. Layer of homogeneous solution of bovine collagen I with collagen concentration of 0.5 wt. % and 50 or 70 wt. % per dry collagen weight of nano hydroxyapatite (nHAP) in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 5 mm exhibiting controlled pore size of 400 μm in the bottom layer 2 mm thick as well as in the top layer 3 mm thick exhibiting pore size of 350 μm and adhering to the well walls. Even after swelling, the substrate remains attached to the well bottom without detaching and floating. This substrate is, thus, suitable for seeding and culturing broad range of cell types and has already been subject to in-vitro and in-vivo tests (FIG. 4-a,b,e,d). Porosity in the bottom layer was equal to 99.3% and was ranging between 97 and 98% for the top collagen/nHAP layer depending on the nHAP content. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 5

Wells in a 96-well plate were layer by layer filled with a biocompatible material in the following sequence:
1. Ultrapure water (Milipore, purity II)
2. Layer of homogeneous solutions of bovine collagen I, concentration of 2 wt. % in ultrapure water
3. Layer of homogeneous solution of bovine collagen I with collagen concentration of 0.5 wt. % and 50 wt. % per dry collagen weight of nano hydroxyapatite (nHAP) in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 4 mm exhibiting controlled pore size of 100 μm in the bottom layer 1 mm thick as well as in the top layer 3 mm thick exhibiting pore size of 350 μm and adhering to the well walls. Even after swelling, the substrate remains attached to the well bottom without detaching and floating. This substrate is, thus, suitable for seeding and culturing broad range of cell types and has already been subject to in-vitro and in-vivo tests (FIG. 4-c,f). Porosity in the bottom layer was equal to 98.5% and was 98% for the top collagen/nHAP layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 6

Wells in a 96-well plate were layer by layer filled with a biocompatible material in the following sequence:
1. Ultrapure water (Milipore, purity II)
2. Layer of homogeneous solutions of bovine collagen I, concentration of 2 wt. % in ultrapure water
3. Layer of homogeneous solution of bovine collagen I with collagen concentration of 0.5 wt. % and 10 wt. % per dry collagen weight of hyaluronic acid (HA) in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 4 mm exhibiting controlled pore size of 400 μm in the bottom layer 3 mm thick as well as in the top layer 1 mm thick exhibiting pore size of 520 μm and adhering to the well walls. Even after swelling, the substrate remains attached to the well bottom without detaching and floating. This substrate is, thus, suitable for seeding and culturing broad range of cell types and has already been subject to in-vitro and in-vivo tests (FIG. 5a). Porosity in the bottom layer was equal to 99.5% and was 98.5% for the top collagen/HA layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 7

Wells in a 48-well plate were layer by layer filled with a biocompatible material in the following sequence:
1. Ultrapure water (Milipore, purity II)
2. Layer of homogeneous solutions of bovine collagen I, concentration of 2 wt. % in ultrapure water
3. Layer of homogeneous solution of bovine collagen I with collagen concentration of 0.5 wt. % and 20 wt. % per dry collagen weight of chitosan in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 8 mm exhibiting controlled pore size of 100 μm in the bottom layer 2 mm thick as well as in the top layer 6 mm thick exhibiting pore size of 600 μm and adhering to the well walls. Even after swelling, the substrate remains attached to the well bottom without detaching and floating. This substrate is, thus, suitable for seeding and culturing broad range of cell types and has already been subject to in-vitro and in-vivo tests (FIG. 5b). Porosity in the bottom layer was equal to 98.5% and was 98% for the top collagen/chitosan layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 8

Wells in a 24-well plate were layer by layer filled with a biocompatible material in the following sequence:
1. Ultrapure water (Milipore, purity II)
2. Layer of homogeneous solutions of bovine collagen I, concentration of 0.5 wt. % in ultrapure water
3. Layer of homogeneous solution of bovine collagen I with collagen concentration of 0.5 wt. % and 30 wt. % per dry collagen weight of oxidized cellulose in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 10 mm exhibiting controlled pore size of 400 μm in the bottom layer 4 mm thick as well as in the top layer 6 mm thick exhibiting pore size of 350 μm and adhering to the well walls. Even after swelling, the substrate remains attached to the well bottom without detaching and floating. This substrate is, thus, suitable for seeding and culturing broad range of cell types and has already been subject to in-vitro and in-vivo tests (FIG. 6a). Porosity in the bottom layer was equal to 99.5% and was 98.3% for the top collagen/chitosan layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 9

Wells in a 96-well plate were layer by layer filled with a biocompatible material in the following sequence:
1. Ultrapure water (Milipore, purity II)
2. Layer of homogeneous solutions of bovine collagen I, concentration of 2 wt. % in ultrapure water
3. Layer of homogeneous solution of bovine collagen I with collagen concentration of 0.5 wt. % and 15 wt. % per dry collagen weight of blood platelet lysate in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 6 mm exhibiting controlled pore size of 400 μm in the bottom layer 3 mm thick as well as in the top layer 3 mm thick exhibiting pore size of 420 μm and adhering to the well walls. Even after swelling, the substrate remains attached to the well bottom without detaching and floating. This substrate is, thus, suitable for seeding and culturing broad range of cell types and has already been subject to in-vitro and in-vivo tests (FIG. 6b). Porosity in the bottom layer was equal to 99.5% and was 99% for the top collagen/chitosan layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 10

Wells in a 24-well plate were layer by layer filled with a biocompatible material in the following sequence:
1. Ultrapure water (Milipore, purity II)
2. Layer of homogeneous solutions of bovine collagen I, concentration of 0.5 wt. % in ultrapure water
3. Layer of homogeneous solution of bovine collagen I with collagen concentration of 0.5 wt. % and 20 wt. % per dry collagen weight of chitin and 10 wt. % per dry collagen weight of blood platelet lysate in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 10 mm exhibiting controlled pore size of 400 μm in the bottom layer 3 mm thick as well as in the top layer 7 mm thick exhibiting pore size of 380 μm and adhering to the well walls. Even after swelling, the substrate remains attached to the well bottom without detaching and floating. This substrate is, thus, suitable for seeding and culturing broad range of cell types and has already been subject to in-vitro and in-vivo tests (FIG. 6b). Porosity in the bottom layer was equal to 99.5% and was 98.5% for the top collagen/chitosan/BPL layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 11

Wells in a 96-well plate were layer by layer filled with a biocompatible material in the following sequence:
1. Ultrapure water (Milipore, purity II)
2. Layer of homogeneous solutions of bovine collagen I, concentration of 2 wt. % in ultrapure water
3. Layer of homogeneous solution of bovine collagen I with collagen concentration of 0.5 wt. % and 20 wt. % per dry collagen weight of oxidized cellulose and 10 wt. % per dry weight of collagen of blood platelet lysate in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 6 mm exhibiting controlled pore size of 400 μm in the bottom layer 2 mm thick as well as in the top layer 4 mm thick exhibiting pore size of 250 μm and adhering to the well walls. Even after swelling, the substrate remains attached to the well bottom without detaching and floating. This substrate is, thus, suitable for seeding and culturing broad range of cell types and has already been subject to in-vitro and in-vivo tests (FIG. 6b). Porosity in the bottom layer was equal to 99.5% and was 98% for the top collagen/OC/BPL layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate (FIG. 7). Standard deviation of the measurements was less than 10% of the average value.

### Example 12

Wells in a 24-well plate were layer by layer filled with a biocompatible material in the following sequence:
1. Ultrapure water (Milipore, purity II)
2. Layer of homogeneous solutions of decellularized collagen tissue, concentration of 0.7 wt. % containing remnants of bioactive proteins in ultrapure water
3. Layer of homogeneous solutions of decellularized collagen tissue, concentration of 0.5 wt. % containing remnants of bioactive proteins in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. The wells contain homogeneous 3D porous substrate with the total thickness of 10 mm exhibiting controlled pore size of 300 μm in the bottom layer 2 mm thick as well as in the top layer 8 mm thick exhibiting pore size of 400 μm and adhering to the well walls. Even after swelling, the substrate remains attached to the well bottom without detaching and floating. This substrate is, thus, suitable for seeding and culturing broad range of cell types and has already been subject to in-vitro and in-vivo tests (FIG. 6b). Porosity in the bottom layer was equal to 88.5% and was 91% for the top layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 13

Wells in a 24-well plate were layer by layer filled with a biocompatible material in the following sequence:
1. Ultrapure water (Milipore, purity II)
2. Layer of homogeneous solutions of decellularized collagen tissue, concentration of 0.7 wt. % containing remnants of bioactive proteins in ultrapure water
3. Layer of homogeneous solutions of decellularized collagen tissue, concentration of 0.5 wt. % containing remnants of bioactive proteins and enriched with 2 □g TGF-β growth factor in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. The wells contain homogeneous 3D porous substrate with the total thickness of 10 mm exhibiting controlled pore size of 300 μm in the bottom layer 5 mm thick as well as in the top layer 5 mm thick exhibiting pore size of 400 μm and adhering to the well walls. Even after swelling, the scaffold remains attached to the well bottom without detaching and floating. This substrate is, thus, suitable for seeding and culturing broad range of cell types and has already been subject to in-vitro and in-vivo tests. Porosity in the bottom layer was equal to 88.5% and was 91% for the top layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 14

Wells in a 24-well plate were layer by layer filled with a biocompatible material in the following sequence:
1. Ultrapure water (Milipore, purity II)
2. Layer of homogeneous solutions of collagen I, concentration of 0.5 wt. % in ultrapure water
3. Layer of randomly oriented nanofibers from halloysite modified gelatine 180 nm in diameter with 20 wt. % areal concentration in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 4 mm exhibiting controlled pore size of 400 μm in the bottom layer 2 mm thick as well as in the top layer 2 mm thick exhibiting pore size of 50 μm and adhering to the well walls. Even after swelling, the substrate remains attached to the well bottom without detaching and floating. This substrate is, thus, suitable for seeding and culturing broad range of cell types and has already been subject to in-vitro and in-vivo tests. Porosity in the bottom layer was equal to 99.3% and was 75% for the top layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 15

Wells in a 48-well plate were layer by layer filled with a biocompatible material in the following sequence:
1. Ultrapure water (Milipore, purity II)
2. Layer of micellar gel from PLA-PGA-PEG block copolymer, concentration of 1 wt. % in ultrapure water
3. Layer of homogeneous chitosan solution, concentration of 2 wt. % in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. The wells contain homogeneous 3D porous substrate with the total thickness of 4 mm exhibiting controlled pore size of 400 μm in the bottom layer 2 mm thick as well as in the top layer 2 mm thick exhibiting pore size of 380 μm and adhering to the well walls. Even after swelling, the substrate remains attached to the well bottom without detaching and floating. This substrate is, thus, suitable for seeding and culturing broad range of cell types and has already been subject to in-vitro and in-vivo tests. Porosity in the bottom layer was equal to 98.2% and was 98.8% for the top layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 16

Wells in a 24-well plate were layer by layer filled with a biocompatible material in the following sequence:
1. Ultrapure water (Milipore, purity II)
2. Layer of homogeneous solutions of decellularized collagen tissue, concentration of 0.7 wt. % containing remnants of bioactive proteins in ultrapure water
3. Layer of randomly oriented nanofibers from halloysite modified gelatine 180 nm in diameter with 20 wt. % areal concentration in ultrapure water
4. Layer of homogeneous solutions of collagen I, concentration of 0.5 wt. % in ultrapure water

5. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 4.5 mm exhibiting controlled pore size of 400 μm in the bottom layer 2 mm thick as well as in the top layer 2 mm thick exhibiting pore size of 380 μm and adhering to the well walls. The middle layer 0.5 mm thick separating the top and bottom layers exhibited pore size of 80 μm. Even after swelling, the substrate remains attached to the well bottom without detaching and floating. This substrate is, thus, suitable for seeding and culturing broad range of cell types and has already been subject to in-vitro and in-vivo tests. Porosity in the bottom layer was equal to 90.3%, 99.3% for the top layer and 75% for the nanofibrous layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 17

Wells in a 96-well plate were layer by layer filled with a biocompatible material in the following sequence:
1. Ultrapure water (Milipore, purity II)
2. Layer of homogeneous collagen solution, concentration of 2 wt. % in ultrapure water
3. Layer of of 2 wt. % gum-karraya modified with 20 wt. % of oxidized cellulose per weight of gum-karaya in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 4 mm exhibiting controlled pore size of 400 μm in the bottom layer 2 mm thick as well as in the top layer 2 mm thick exhibiting pore size of 150 μm and adhering to the well walls. Even after swelling, the substrate remains attached to the well bottom without detaching and floating. This substrate is, thus, suitable for seeding and culturing broad range of cell types and has already been subject to in-vitro and in-vivo tests. Porosity in the bottom layer was equal to 98.5% and 98% for the top layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 18

Wells in a 96-well plate were layer by layer filled with a biocompatible material in the following sequence:
1. Ultrapure water (Milipore, purity II)
2. Layer of homogeneous collagen solution, concentration of 2 wt. % in ultrapure water
3. Layer of 2 wt. % homogeneous solution of chitosan in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 5 mm exhibiting controlled pore size of 400 μm in the bottom layer 2.5 mm thick as well as in the top layer 2.5 mm thick exhibiting pore size of 380 μm and adhering to the well walls. Even after swelling, the substrate remains attached to the well bottom without detaching and floating. This substrate is, thus, suitable for seeding and culturing broad range of cell types and has already been subject to in-vitro and in-vivo tests. Porosity in the bottom layer was equal to 99.5% and 98% for the top layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

### Example 19

Wells in a 96-well plate were layer by layer filled with a biocompatible material in the following sequence:
1. Ultrapure water (Milipore, purity II)
2. Layer of homogeneous collagen solution, concentration of 2 wt. % in ultrapure water
3. Layer of 2 wt. % homogeneous suspension of silk fibroin in ultrapure water
4. Ultrapure water (Milipore, purity II).

The materials in the wellplate were frozen under controlled conditions after deposition of each layer first at −18° C. (1 hour), then at −35° C. (1 hour), and, finally, freeze dried at the condensator temperature of −95° C. and pressure lower than 1000 Pa to constant weight. Following freeze drying, collagen is cross-linked using EDC/NHS system directly in the well and, again, freeze dried under the same conditions as described above. The wells contain homogeneous 3D porous substrate with the total thickness of 4 mm exhibiting controlled pore size of 400 μm in the bottom layer 2.5 mm thick as well as in the top layer 1.5 mm thick exhibiting pore size of 380 μm and adhering to the well walls. Even after swelling, the scaffold remains attached to the well bottom without detaching and floating. This substrate is, thus, suitable for seeding and culturing broad range of cell types and has already been subject to in-vitro and in-vivo tests. Porosity in the bottom layer was equal to 99.5% and 97.6% for the top layer. Porosity was determined by image analysis of the 3D morphology of the freeze dried substrate obtained employing the microCT computer tomograph (for example Nano3DX Rigaku, Japan). The porosities shown above are the average values calculated from 5 repetitive measurements performed for randomly selected wells from the wellplate. Standard deviation of the measurements was less than 10% of the average value.

1. A carrier for cell culturing, which contains a multiwell plate, wherein at least one of the wells of the said multiwell plate contains a porous substrate, preferably having the porosity of ≥90% and adapted for cell culturing, and wherein the said porous substrate adheres to the surface of the said well.

2. The carrier according to claim 1, wherein the porous substrate is provided in at least 25% of the wells, more preferably in at least 50% of the wells or in all the wells of the multiwell plate.

3. The carrier according to claim 1, wherein the multiwell plate is made of a material selected from the group consisting of plastics, glass, ceramics, metals, and combinations of these materials.

4. The carrier according to claim 1, wherein the porous substrate is composed of at least two layers which differ mutually in at least one of: material composition, presence or absence of additives, composition of additives, porosity, pore size, pore connectivity.

5. The carrier according to claim 1, wherein the pore size of the porous substrate is within the range from 0.1 to 1000 μm, preferably between 5 and 1000 μm or between 100 and 2000 nm or within the range from 50 to 600 μm.

6. The carrier according to claim 1, wherein the material of the porous substrate is selected from the group consisting of proteins of the extracellular matrix (ECM); structural proteins such as collagens, fibrin, silk fibroin, elastin or gelatine; polysaccharides such as hyaluronic acid and its derivatives, chitosan and its derivatives, starches, resinous gums, cellulose and its derivatives; resins; biocompatible synthetic polymers, such as polylactic acid, polyglycolic acid, polyethyleneglycol and their block copolymers, polycaprolaktone, polyhydroxybutyrate, polyurethanes; natural inorganic nanotubes; and combinations of these materials; wherein the porous substrate material optionally is cross-linked, and/or the porous substrate material optionally contains as additives: bioactive proteins, their fragments or mixtures, phosphates, organic or inorganic nano-particles such as hydroxyapatite, alpha- or beta-tricalcium phosphate, oxide ceramics, such as SiO₂, ZnO, metal nanoparticles such as Ag, Au, Mg, or metal nano-particles decorated with covalently or physically bound organic ligands.

7. The carrier according to claim 1, wherein the porous substrate has the smallest dimension of at least 10 micrometers, preferably at least 100 micrometers, more preferably at least 0.5 mm, most preferably at least 1 to 10 mm.

8. A method for preparation of a carrier for cell culturing, the said method comprising the following sequence of steps:
applying a layer of solvent into at least one well of the multiwell plate and freezing the layer of solvent,
applying at least one layer of a material for a porous substrate into the said at least one well of the multiwell plate, and freezing each layer after it is applied,
applying a cover layer of the solvent into the said at least one well of the multiwell plate, followed by freezing the cover layer, and
freeze drying after applying each layer separately, or after applying all the layers into the said at least one well.

9. The method according to claim 8, wherein the the freezing is performed in two steps: first freezing to a temperature between −5 and −40° C., followed by freezing to a temperature between −20 and −60° C.

10. The method according to claim 8, wherein freeze drying is carried out at a temperature lower than −70° C., preferably at lower than atmospheric pressure, more preferably at a pressure of 1000 Pa or less.

11. The method according to claim 8, wherein the material of the porous substrate is applied in the form of a solution, suspension, aerogel, emulsion, hydrogel, microparticles or nano-fibers.

12. The method according to claim 8, wherein the material of the porous substrate is cross-linked using a cross-linking agent.

13. A method of culturing cells, comprising the steps of:
providing a carrier according to claim 1,
seeding cells on the porous substrates in the said carrier,
culturing cells on the the porous substrates of the said carrier,
optionally subjecting the cultured cells to a chemical, physical or biological analysis.
